(19) United States
(12) Patent Application Publication
(10) Pub. No.: US 2018/0168991 A1
(43) Pub. Date: Jun. 21, 2018

(54) STYLING AGENT WITH IMPROVED LONG-TERM HOLD AND DEGREE OF HOLD

(71) Applicant: Henkel AG & Co. KGaA, Duesseldorf (DE)

(72) Inventors: Julia Bibiane Lange, Bad Bramstedt (DE); Rene Scheffler, Ellerau (DE); Diane Metten, Hamburg (DE); Cyrielle Martinez, Hamburg (DE)

(73) Assignee: Henkel AG & Co. KGaA, Duesseldorf (DE)

(21) Appl. No.: 15/847,931

(22) Filed: Dec. 20, 2017

(30) Foreign Application Priority Data
Dec. 20, 2016 (DE) 10 2016 225 665.3

(51) Int. Cl. A61K 8/81; A61Q 5/06

(52) U.S. Cl. CPC A61K 8/8152; A61K 8/8176; A61K 2800/48; A61Q 5/06; A61K 2800/594; A61K 8/8147

(57) ABSTRACT

The present disclosure relates to a cosmetic agent for temporary deformation of hair, containing a combination of a selected copolymer and a vinylpyrrolidone homopolymer. The cosmetic agent provides an extremely good combination of stiffness and long-term hold.

### CROSS-REFERENCE TO RELATED APPLICATION

This application claims priority to German Patent Application No. 10 2016 225 665.3, filed Dec. 20, 2016, which is incorporated herein by reference in its entirety.

### TECHNICAL FIELD

The present disclosure relates to a cosmetic agent for temporary deformation of keratinous fibers, for example human hair, wherein the agent contains a combination of a selected copolymer and a vinylpyrrolidone homopolymer.

### BACKGROUND

The temporary design of hairstyles for a longer period of time of up to several days normally includes the use of firming active ingredients. Therefore, hair treatment agents that temporarily shape the hair play a useful role.

Corresponding agents for temporary deformation may contain synthetic polymers and/or waxes as a firming active ingredient. Agents aiding the temporary deformation of keratinous fibers can be packaged, for instance, as hairspray, hair wax, hair gel or mousse.

One feature of an agent for temporary deformation of hair, also referred to hereinafter as a styling agent, includes giving the treated fibers in the newly modeled shape—i.e. a shape impressed on the hair—the strongest possible hold. This is also referred to as a strong hairstyle hold or a high degree of hold of the styling agent. The hairstyle hold may be determined by the type and amount of firming active ingredients used, wherein the other components of the styling agent can also have an influence.

In addition to a high degree of hold, the styling agent may fulfill a series of additional requirements. They can be roughly divided in to characteristics on the hair, characteristics of the respective formulation, e.g. characteristics of the foam, gel or sprayed aerosol, and characteristics relating to the handling of the styling agent. This includes, for example, moisture resistance, low stickiness (tack) and a balanced conditioning effect.

Furthermore, a styling agent may be universally applicable for all hair types, insofar as possible, and mild to the hair and skin.

In order to satisfy the various requirements, a multitude of synthetic polymers were developed as firming active ingredients, which can be used in styling agents. The polymers can be divided into cationic, anionic, nonionic and amphoteric firming polymers. In some instances, the polymers create a polymer film when used on the hair, which lends the hairstyle a strong hold and is sufficiently flexible in order to not break under stress. If the polymer film is too brittle, so-called flakes form, i.e. residue, which detach during movement of the hair and give the impression that the user of the corresponding styling agent has dandruff. Similar problems arise when waxes are used as a firming active ingredient in the styling agent. If the styling agent is a gel or a paste, the polymers might also have thickening properties.

Known anionic polymers that are used in hair fixing products are hydrophobically-modified acrylate copolymers. These are commercially available (INCI: acrylate copolymer (and) water) and act as a thickening agent

Known nonionic polymers that are used in hair fixing products are polyvinylpyrrolidones (PVP). These are homopolymers of vinylpyrrolidone. Vinylpyrrolidone homopolymers are, for example, sold under the name Luviskol® (BASF) and are used as film formers and/or thickening agents. In particular for use in cosmetic agents and styling products as film formers or thickening agents, the Luviskol® K product range (BASF), in which vinylpyrrolidone homopolymers are offered with different molecular weights, is available. From WO2016/030049 A1 a styling agent is known, for example, which contains an anionic acrylate resin and polyvinylpyrrolidone.

### BRIEF SUMMARY

An exemplary cosmetic agent for temporary deformation of keratinous fibers contains:

(a) at least one copolymer A, including
at least one monomer A1 selected from acrylic acid, methacrylic acid, acrylic acid alkyl esters and methacrylic acid alkyl esters, and
at least one amphoteric monomer A2 of the Formula (I)

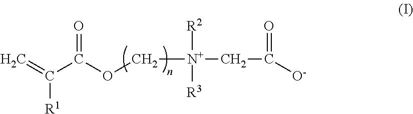

wherein R¹ for H or CH₃, R² and R³, each independently of one another, denote optionally branched C₁₋₁₀ alkyl, and n denotes an integer from 1 to 20, and

(b) at least one vinylpyrrolidone homopolymer.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure addresses the problem of providing additional polymer combinations, which is exemplified by good film-forming and/or fixing properties, has a very high degree of hold without the need to dispense with flexibility and good moisture resistance—particularly sweat and water resistance—and is also suitable for production of stable viscous and stable transparent cosmetic agents. In particular, currently available styling agents can be improved, because a good combination of stiffness and long-term hold is not always sufficiently guaranteed. Therefore, the present disclosure addresses the problem of preparing such styling agents that provide the aforementioned properties, particularly with regard to good stiffness and a good, long-term hold.

This problem was solved by a cosmetic agent for temporary deformation of keratinous fibers, which contains:

(a) at least one copolymer A, including:
at least one monomer A1 selected from acrylic acid, methacrylic acid, Acrylic acid alkyl esters and methacrylic acid alkyl esters, and
at least one amphoteric monomer A2 of the Formula (I)

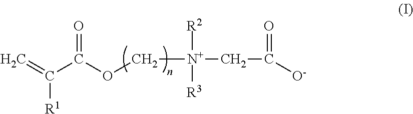

Wherein R¹ for H or CH₃, R² and R³, each independently of one another, denote optionally branched C₁₋₁₀ alkyl, and n denotes an integer from 1 to 20, and
(b) at least one vinylpyrrolidone homopolymer.

Surprisingly, in the context of the present disclosure, it was discovered that a combination of these two polymers can achieve an improved combination of long-lasting effect and stiffness of styling products, in particular of hair gels.

As contemplated herein, the term keratinous fibers comprises fur, wool and feathers, and in many instances human hair.

The first component (a) of the cosmetic agent is copolymer A, which contains at least one monomer A1 and at least one monomer A2.

The monomer A1 is selected from acrylic acid, methacrylic acid, acrylic acid alkyl esters and methacrylic acid alkyl esters. Exemplary monomers A1 are acrylic acid, methacrylic acid, acrylic acid-C₁₋₂₀ alkyl esters and methacrylic acid-C₁₋₂₀ alkyl ester.

Consideration is given to monomer A1 selected from acrylic acid, methacrylic acid, acrylic acid methyl ester, methacrylic acid methyl ester, acrylic acid ethyl ester, methacrylic acid ethyl ester, acrylic acid propyl ester, methacrylic acid propyl ester, acrylic acid isopropyl ester, methacrylic acid isopropyl ester, acrylic acid butyl ester, methacrylic acid butyl ester, acrylic acid tert. butyl ester, methacrylic acid tert. butyl ester, acrylic acid sec. butyl ester, methacrylic acid sec. butyl ester, acrylic acid pentyl ester, methacrylic acid pentyl ester, acrylic acid hexyl ester, hexyl ester, acrylic acid heptyl ester, methacrylic acid heptyl ester, acrylic acid octyl ester, methacrylic acid octyl ester, acrylic acid nonyl ester, methacrylic acidn onyl ester, acrylic acid decyl ester, methacrylic acid decyl ester, acrylic acid lauryl ester, methacrylic acid lauryl ester, acrylic acid myristyl ester, methacrylic acid myristyl ester, acrylic acid cetyl ester, methacrylic acid cetyl ester, acrylic acid stearyl ester and methacrylic acid stearyl ester. The monomer A1 selected from acrylic acid, methacrylic acid, acrylic acid methyl ester, methacrylic acid methyl ester, acrylic acid ethyl ester, methacrylic acid ethyl ester, acrylic acid lauryl ester, methacrylic acid lauryl ester, acrylic acid stearyl ester and methacrylic acid stearyl ester is exemplary.

Exemplary monomers A2 are (meth)acryloyl alkyl betaines of the Formula (I), wherein R² and R³, independently of one another, denote methyl, ethyl, propyl, isopropyl, butyl, isobutyl or tert.-butyl, for example methyl.

Exemplary monomers A2 are also selected from (meth)acryloyl alkyl betaines of the Formula (I), wherein n denotes an integer from 1 to 5, for example an integer from 1 to 3 and most preferably 2.

Consideration is also given to monomers A2 selected from (meth)acryloyl alkyl betaines of the Formula (I), wherein R¹ denotes CH₃.

Consideration is given to the monomers A2 selected from (meth)acryloyl alkyl betaines of the Formula (I), wherein R² and R³, independently of one another, denote methyl, ethyl, propyl, isopropyl, butyl, isobutyl or tert.-butyl, for example methyl, n denotes an integer from about 1 to about 5, such as an integer from about 1 to about 3 and for example 2, and R¹ denotes CH₃.

Consideration is given to monomer A2 selected from (meth)acryloyl alkyl betaines of the Formula (I), wherein R¹, R² and R³ denote CH₃, and n denotes 2.

For all previously described embodiments, it is contemplated that the copolymer A (for example exclusively) includes at least two monomers A1 and at least one (meth)acryloyl alkyl betaine.

In one embodiment, the copolymer A (for example exclusively) includes at least two different monomers A1 and methacryloyl ethyl betaine as monomer A2.

An exemplary copolymer A with INCI name methacryloyl ethyl betaine/acrylate copolymer that can be used is sold under the name Advantage PG 30 by the company Ashland.

The agents may contain the copolymer A in a quantity of from about 1 to about 20 wt. %, such as from about 5 to about 15 wt. % and for example from about 8 to about 12 wt. %, relative to the total agent.

The agents can, of course, also contain multiple different copolymers A, wherein the total amount of copolymer A, relative to the total agent, does not exceed 20 wt. %.

The copolymers A can be produced from the aforementioned monomers using the known polymerization methods and are generally commercially available.

The agent can contain a vinylpyrrolidone homopolymer as component (b).

It is contemplated that the vinylpyrrolidone homopolymers may be selected from vinylpyrrolidone homopolymers with a K value (1 wt. % solution of PVP, Brookfield at 23° C.) in water of from about 20 to about 100. A K value of from about 80 to about 100 is exemplary, and 90 is also exemplary. The K value, which is also referred to as the intrinsic viscosity, is a parameter used to characterize polymers that is easily derived from the relative viscosity by employing viscosity measurements.

Exemplary vinylpyrrolidone homopolymers are available under the trade names Luviskol® K 30, Luviskol® K 80, Luviskol® K 85, and Luviskol® K 90 from the company BASF. SE. Luviskol® K 90 is most preferred. Luviskol® K90 is a 20% aqueous, colorless to slightly yellow solution of polyvinylpyrrolidone. The product has a K value of from about 90.0 to about 98.0 (1% (m/V) in water), a solids content of from about 19.0 to about 21.0 wt. % and a pH value of from about 7.0 to about 9.0 (10 wt. % solids content in water).

The cosmetic agent contains the vinylpyrrolidone homopolymer (b), relative to the total weight of the cosmetic agent, e.g. in a quantity of from about 0.1 to about 10 wt. %, such as from about 0.5 to about 8 wt. %, for example from about 1 to about 5 wt. %, relative to the total weight of the agent in each case.

In an embodiment of the present disclosure, the cosmetic agent contains, as vinylpyrrolidone homopolymer (b), the homopolymer that is commercially available under the name Luviskol® K90 (BASF) and, as copolymer A (a), the copolymer that is commercially available under the name Advantage PG 30 (Ashland). Good results with regard to a combination of stiffness and long-term hold were achieved with this combination. This polymer combination is useful for styling products in gel form.

Additional properties generally included in styling products, such as moisture resistance and low stickiness, are also provided with this combination, particularly when packaged as hair gel.

The cosmetic agent of the present disclosure may contain one or more additional components that act as a thickening agent or gel former, which are different from the copolymer A and vinylpyrrolidone homopolymer (b) and also aid film formation.

Examples are cationic, anionic, nonionic or amphoteric polymers. The proportion by weight of these additional components to the total weight of the cosmetic agent can be comparatively low due to the present of components (a) and (B) and, for example, be from about 0.02 to about 1.5 wt. %, such as from about 0.05 to about 1 wt. % and for example from about 0.1 to about 0.7 wt. %.

Examples are acrylamide/ammonium acrylate copolymer, acrylamides/DMAPA acrylates/methoxy PEG methacrylate copolymer, acrylamidopropyltrimonium chloride/acrylamide copolymer, acrylamidopropyltrimonium chloride/acrylates copolymer, acrylates/acetoacetoxyethyl methacrylate copolymer, acrylates/acrylamide copolymer, acrylates/ammonium methacrylate copolymer, acrylates/t-butylacrylamide copolymer, acrylates copolymer, acrylates/C1-2 succinates/hydroxyacrylates copolymer, acrylates/lauryl acrylate/stearyl acrylate/ethylamine oxide methacrylate copolymer, acrylates/octylacrylamide copolymer, acrylates/octylacrylamide/diphenyl amodimethicone copolymer, acrylates/stearyl acrylate/ethylamine oxide methacrylate copolymer, acrylates/CA copolymer, acrylates/VP copolymer, adipic acid/diethylenetriamine copolymer, adipic acid/dimethylaminohydroxypropyl diethylenetriamine copolymer, adipic acid/epoxypropyl diethylenetriamine copolymer, adipic acid/isophthalic acid/neopentyl glycol/trimethylolpropane copolymer, allyl stearate/VA copolymer, aminoethylacrylate phosphate/acrylates copolymer, aminoethylpropanediol-acrylates/acrylamide copolymer, aminoethylpropanediol-AMPD-acrylates/diacetoneacrylamide copolymer, ammonium va/acrylates copolymer, AMPD-acrylates/diacetoneacrylamide copolymer, AMP-acrylates/allyl methacrylate copolymer, AMP-acrylates/C1-18 alkyl acrylates/C1-8 alkyl acrylamide copolymer, AMP-acrylates/diacetoneacrylamide copolymer, AMP-acrylates/dimethylaminoethylmethacrylate copolymer, bacillus/rice bran extract/soybean extract ferment filtrate, bis-butyloxyamodimethicone/PEG-60 copolymer, butyl acrylate/ethylhexyl methacrylate copolymer, butyl acrylate/hydroxypropyl dimethicone acrylate copolymer, butylated PVP, butyl ester of ethylene/MAcopolymer, butyl ester of PVM/MA copolymer, calcium/sodium PVM/MA copolymer, corn starch/acrylamide/sodium acrylate copolymer, diethyleneglycolamine/epichlorohydrin/piperazine copolymer, dimethicone crosspolymer, diphenyl amodimethicone, ethyl ester of PVM/MA copolymer, hydrolyzed wheat protein/PVP crosspolymer, isobutylene/ethylmaleimide/hydroxyethylmaleimide copolymer, isobutylene/MAcopolymer, isobutylmethacrylate/bis-hydroxypropyl dimethicone acrylate copolymer, isopropyl ester of PVM/MA copolymer, lauryl acrylate crosspolymer, lauryl methacrylate/glycol dimethacrylate crosspolymer, MEA-sulfite, methacrylic acid/sodium acrylamidomethyl propane sulfonate copolymer, methacryloyl ethyl betaine/acrylates copolymer, octylacrylamide/acrylates/butylaminoethyl methacrylate copolymer, PEG/PPG-25/25 dimethicone/acrylates copolymer, PEG-8/SMDI copolymer, polyacrylamide, polyacrylate-6, polybeta-alanine/glutaric acid crosspolymer, polybutylene terephthalate, polyester-1, polyethylacrylate, polyethylene terephthalate, polymethacryloyl ethyl betaine, polypentaerythrityl terephthalate, polyperfluoroperhydrophenanthrene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-22, polyquaternium-24, polyquaternium-27, polyquaternium-28, polyquaternium-29, polyquaternium-30, polyquaternium-31, polyquaternium-32, polyquaternium-33, polyquaternium-34, polyquaternium-35, polyquaternium-36, polyquaternium-37, polyquaternium-39, polyquaternium-45, polyquaternium-46, polyquaternium-47, polyquaternium-48, polyquaternium-49, polyquaternium-50, polyquaternium-55, polyquaternium-56, polysilicone-9, polyurethane-1, polyurethane-6, polyurethane-10, polyvinyl acetate, polyvinyl butyral, polyvinylcaprolactam, polyvinylformamide, polyvinyl imidazolinium acetate, polyvinyl methyl ether, potassium butyl ester of pvm/ma copolymer, potassium ethyl ester of PVM/MA copolymer, PPG-70 polyglyceryl-10 ether, PPG-12/SMDI copolymer, ppg-51/SMDIcopolymer, PPG-10 sorbitol, PVM/MA copolymer, PVP, PVP/VA/itaconic acid copolymer, PVP/VA/vinyl propionate copolymer, rhizobian gum, rosin acrylate, shellac, sodium butyl ester of PVM/MA copolymer, sodium ethyl ester of PVM/MA copolymer, sodium polyacrylate, sterculia urens gum, terephthalic acid/isophthalic acid/sodium isophthalic acid sulfonate/glycol copolymer, trimethylolpropane triacrylate, trimethylsiloxysilylcarbamoyl pullulan, VA/crotonates copolymer, VA/crotonates/methacryloxybenzophenone-1 copolymer, VA/crotonates/vinyl neodecanoate copolymer, VA/crotonates/vinyl propionate copolymer, VA/DBM copolymer, VA/vinyl butyl benzoate/crotonates copolymer, vinylamine/vinyl alcohol copolymer, vinyl caprolactam/VP/dimethylaminoethyl methacrylate copolymer, VP/acrylates/lauryl methacrylate copolymer, VP/dimethylaminoethylmethacrylate copolymer, VP/DMAPA acrylates copolymer, VP/hexadecene copolymer, VP/VA copolymer, VP/vinyl caprolactam/DMAPA acrylates copolymer, yeast palmitate and styrene/VP copolymer.

Examples of nonionic polymers are:
vinylpyrrolidone/vinylester copolymers, which are sold, for example, under the name Luviskol (BASF). Luviskol VA 64 and Luviskol VA 73, in each case vinylpyrrolidone/vinylacetate-copolymers, are preferable nonionic polymers,
cellulose ethers, such as hydroxypropyl cellulose, hydroxyethyl cellulose and methylhydroxypropyl cellulose, are sold, for example, under the names Culminal and Benecel (AQUALON),
shellac,
siloxanes, which can be water-soluble or non-water-soluble. Fluid and non-fluid siloxanes are suitable, wherein non-fluid siloxanes are to be understood to mean such compounds having a boiling point above 200° C. under normal pressure. Suitable siloxanes are polydialkylsiloxanes, such as polydimethylsiloxane, polyalkylarylsiloxanes, such as polyphenylmethylsiloxane, ethoxylated polydialkylsiloxanes and polydialkylsiloxanes which contain amine and/or hydroxyl groups, and
glycosidically substituted silicones. The additional component acting as a gel former may be a polyacrylic acid (INCI: carbomer), which is commercially available under the name Carbopol® in different embodiments. Polyacrylic acid has structural units of the Formula

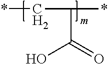

in which m varies depending on the molar mass.

Exemplary agents are exemplified in that they contain polyacryl acids with a molar mass of from about 10 to about 250 kDa, such as from about 25 to about 200 kDa, for example about from about 50 to about 150 kDa and from about 70 to about 100 kDa.

The polyacrylic acid may be contained in a proportion of from about 0.02 to about 1.5 wt. %, such as from about 0.05 to about 1 wt. % and for example from about 0.1 to about 0.7 wt. %, relative to the total weight of the cosmetic agent.

It is also contemplated that the cosmetic agent contains less than 1 wt. %, and for example less than 0.5 wt. %, relative to the total weight of the agent, of a vinylpyrrolidone-containing copolymer.

The cosmetic agent can contain additional conventional ingredients of styling products. Additional care substances, in particular, can be mentioned as additional suitable auxiliary substances and additives.

As the nourishing agent, the agent can contain at least one protein hydrolysate and/or a derivate thereof, for example. Protein hydrolysates are product mixtures obtained through the acidically, basically or enzymatically catalyzed decomposition of proteins. The expression protein hydrolysates also includes total hydrolysates, as well as individual amino acids and the derivatives thereof, as well as mixtures of various amino acids. The molecular weight of the protein hydrolysates usable as contemplated herein is between about 75, the molecular weight for glycine, and about 200.000, such as from about 75 to about 50.000 and for example from about 75 to about 20.000 Dalton. Arginine, for example, is an amino acid that can be used.

The agent can also contain, as a nourishing agent a vitamin, a provitamin, a vitamin precursor and/or a derivative thereof. As contemplated herein, such vitamins, provitamins and vitamin precursors may be assigned to the groups A, B, C, E, F and H.

An exemplary nourishing agent is the provitamin B5, which is also known under the INCI name PANTHENOL. An additional exemplary nourishing agent is vitamin B3, which is also known as nicotinic acid amide or niacin amide.

The addition of provitamin B5 also increases the flexibility of the polymer film formed using the agent.

The agent as contemplated herein can also contain, as a nourishing agent, a plant extract, as well as mono- and/or oligosaccharides and/or lipids.

Furthermore, oil bodies are suitable as care substances. The natural and synthetic cosmetic oil bodies include, for example, vegetable oils, liquid paraffin oils, isoparaffin oils and synthetic hydrocarbons, and di-n-alkyl ethers having a total of between about 12 to about 36 carbon atoms, in particular from about 12 to about 24 carbon atoms. Cosmetic agents preferably used as contemplated herein contain at least one oil body, such as at least one oil body from the group of silicone oils. The group of silicone oils also includes, for example, dimethicones, which also include cyclomethicones, amino-functional silicones and dimethiconols. Dimethicones can be linear or branched, as well as cyclical or cyclical and branched. Suitable silicone oils or silicone gums are, in particular dialkyl- and alkylarylsiloxanes, such as dimethylpolysiloxane and methylphenylpolysiloxane, and alkoxylated, quaternized or anionic derivatives thereof. Cyclical and linear polydialkylsiloxanes, alkoxylated and/or aminated derivatives thereof, dihydroxypolydimethylsiloxanes and polyphenylalkylsiloxanes are exemplary.

Ester oils, i.e. esters of C6-C30 fatty acids with C2-C30 fatty alcohols, such as monoesters of fatty acids with alcohols having from about 2 to about 24 carbon atoms, such as isopropyl myristate (Rilanit® IPM), isononanoic acid c16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate)(Cetiol®, lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V) are additional exemplary care oil bodies.

Furthermore, dicarboxylic acid esters, symmetrical, asymmetrical or cyclic esters of carbonic acid with fatty alcohols, trifatty acid esters of saturated and/or unsaturated linear and/or branched fatty acids with glycerol or fatty acid partial glycerides, which are to be understood as monoglycerides, diglycerides and technical mixtures thereof are suitable as care sub stances.

Furthermore, emulsifiers and/or surfactants may be contained in the agent. PEG derivatives of hydrated castor oil are preferred, which are available, for example, under the name PEG Hydrogenated Castor Oil, such as PEG-30 Hydrogenated Castor Oil, PEG-33 Hydrogenated Castor Oil, PEG-35 Hydrogenated Castor Oil, PEG-36 Hydrogenated Castor Oil or PEG-40 Hydrogenated Castor Oil. The use of PEG-40 Hydrogenated Castor Oil is exemplary. PEG derivatives of hydrated castor oil are suitable in a quantity of from about 0.05 to about 1.5 wt. %, such as from about 0.1 to about 1 wt. %, for example from about 0.2 to about 0.8 wt. %, and from about 0.3 to about 0.6 wt. %, relative to the total amount of the agent in each case.

The agent can also contain a pH control agent. Suitable pH control agents include acids or bases. A suitable pH control agent is aminomethyl propanol, which may be contained in a quantity of from about 0.01 to about 1 wt. %, and for example from about 0.1 to about 0.5 wt. %, relative to the total amount of the agent in each case.

The cosmetic agents contain the ingredients and/or active ingredients in a cosmetically acceptable carrier.

Suitable cosmetically acceptable carriers are aqueous, alcoholic or aqueous-alcoholic media for example having at least 10 wt. % water, relative to the total weight of the agent.

It is contemplated that the cosmetic carrier may contain water, particularly in a quantity that is for example at least 10 wt. %, more such as at least 20 wt %, for example at least 40 wt. % water relative to the total weight of the agent.

Low alcohols having from about 1 to about 4 carbon atoms conventionally used for cosmetic purpose, such as ethanol and isopropanol, can be used, for example as alcohols.

The cosmetic agent of the present disclosure can be packed in the conventional forms for temporary deformation of hair, such as hair gel, hairspray, mousse or hair wax. Packaging as hair gel is suitable.

Hair mousse and hairsprays may require the presence of propellants. As contemplated herein, however, these propellants might not contain any or only minor quantities of hydrocarbons. As contemplated herein, dimethyl ether is a suitable propellant.

The present disclosure also relates to the use of inventive cosmetic agents for temporary deformation of keratinous fibers, for example of human hair, as well as a method for temporary deformation of keratinous fibers, for example human hair, wherein the cosmetic agent is applied on keratinous fibers.

Tabular Overview

A summary of some cosmetic agents is provided in the following tables (specifications in wt. % relative to the total weight of the cosmetic agent, unless otherwise specified).

| | Formula 1 | Formula 2 | Formula 3 |
|---|---|---|---|
| Copolymer A (a) | 1-20 | 5-15 | 8-12 |
| Vinylpyrrolidone | 0.1-10 | 0.5-8 | 1-5 |
| homopolymer (b) | | | |
| Misc | add 100 | add 100 | add 100 |
| | Formula 1a | Formula 2a | Formula 3a |
| Copolymer A, | 1-20 | 5-15 | 8-12 |
| containing at least one | | | |
| monomer A1 selected | | | |
| from acrylic acid, | | | |
| methacrylic acid, acrylic | | | |
| acid alkyl esters and | | | |
| methacrylic acid alkyl | | | |
| esters, and at least one | | | |
| monomer A2, | | | |
| comprising a | | | |
| (meth)acryloyl alkyl | | | |
| betaine. | | | |
| Vinylpyrrolidone | 0.1-10 | 0.5-8 | 1-5 |
| homopolymer (b) | | | |
| Misc | add 100 | add 100 | add 100 |
| | Formula 1b | Formula 2b | Formula 3b |
| Copolymer A, containing | 1-20 | 5-15 | 8-12 |
| at least two monomers A1 | | | |
| selected from acrylic acid, | | | |
| methacrylic acid, acrylic | | | |
| acid alkyl esters and | | | |
| methacrylic acid alkyl | | | |
| esters, and at least one | | | |
| monomer A2, comprising | | | |
| a (meth)acryloyl alkyl | | | |
| betaine. | | | |
| Vinylpyrrolidone | 0.1-10 | 0.5-8 | 1-5 |
| homopolymer (b) | | | |
| Misc | add 100 | add 100 | add 100 |
| | Formula 1c | Formula 2c | Formula 3c |
| Copolymer A: | 1-20 | 5-15 | 8-12 |
| Methacryloyl ethyl | | | |
| betaine/acrylates (INCI) | | | |
| Vinylpyrrolidone | 0.1-10 | 0.5-8 | 1-5 |
| homopolymer (b) | | | |
| Misc | add 100 | add 100 | add 100 |
| | Formula 4 | Formula 5 | Formula 6 |
| Copolymer A (a) | 1-20 | 5-15 | 8-12 |
| Vinylpyrrolidone | 0.1-10 | 0.5-8 | 1-5 |
| homopolymer (b) | | | |
| Polyacrylic acid | 0.02-1.5 | 0.05-1 | 0.1-0.7 |
| Misc | add 100 | add 100 | add 100 |
| | Formula | Formula | Formula |
| | 4a | 5a | 6a |
| Copolymer A, containing | 1-20 | 5-15 | 8-12 |
| at least one monomer A1 | | | |
| selected from acrylic acid, | | | |
| methacrylic acid, acrylic | | | |
| acid alkyl esters and | | | |
| methacrylic acid alkyl | | | |
| esters, and at least one | | | |
| monomer A2, comprising a | | | |
| (meth)acryloyl alkyl | | | |
| betaine. | | | |
| Vinylpyrrolidone | 0.1-10 | 0.5-8 | 1-5 |
| homopolymer (b) | | | |
| Polyacrylic acid | 0.02-1.5 | 0.05-1 | 0.1-0.7 |
| Misc | add 100 | add 100 | add 100 |
| | Formula 4b | Formula 5b | Formula 6b |
| Copolymer A, containing | 1-20 | 5-15 | 8-12 |
| at least one monomer A1 | | | |
| selected from acrylic acid, | | | |
| methacrylic acid, acrylic | | | |
| acid alkyl esters and | | | |
| methacrylic acid alkyl | | | |
| esters, and at least one | | | |
| monomer A2, comprising | | | |
| a (meth)acryloyl alkyl | | | |
| betaine. | | | |
| Vinylpyrrolidone | 0.1-10 | 0.5-8 | 1-5 |
| homopolymer (b) | | | |
| Polyacrylic acid | 0.02-1.5 | 0.05-1 | 0.1-0.7 |
| Misc | add 100 | add 100 | add 100 |
| | Formula 4c | Formula 5c | Formula 6c |
| Copolymer A, containing | 1-20 | 5-15 | 8-12 |
| at least two monomers A1 | | | |
| selected from acrylic | | | |
| acid, methacrylic acid, | | | |
| acrylic acid alkyl esters | | | |
| and methacrylic acid | | | |
| alkyl esters, and at least | | | |
| one monomer A2, | | | |
| comprising a | | | |
| (meth)acryloyl alkyl | | | |
| betaine. | | | |
| Vinylpyrrolidone | 0.1-10 | 0.5-8 | 1-5 |
| homopolymer (b) | | | |
| Polyacrylic acid | 0.02-1.5 | 0.05-1 | 0.1-0.7 |
| Misc | add 100 | add 100 | add 100 |
| | Formula 4d | Formula 5d | Formula 6d |
| Copolymer A: | 1-20 | 5-15 | 8-12 |
| Methacryloyl ethyl | | | |
| betaine/acrylates (INCI) | | | |
| Vinylpyrrolidone | 0.1-10 | 0.5-8 | 1-5 |
| homopolymer (b) | | | |
| Polyacrylic acid | 0.02-1.5 | 0.05-1 | 0.1-0.7 |
| Aminomethyl propanol | 0.01-1 | 0.1-0.5 | 0.1-0.5 |
| Misc | add 100 | add 100 | add 100 |
| | Formula 4e | Formula 5e | Formula 6e |
| Copolymer A: | 1-20 | 5-15 | 8-12 |
| Methacryloyl ethyl | | | |
| betaine/acrylates (INCI) | | | |
| Vinylpyrrolidone | 0.1-10 | 0.5-8 | 1-5 |
| homopolymer (b) | | | |
| Polyacrylic acid | 0.02-1.5 | 0.05-1 | 0.1-0.7 |
| Aminomethyl propanol | 0.01-1 | 0.1-0.5 | 0.1-0.5 |
| PEG-40 Hydrogenated | 0.1-1 | 0.2-0.8 | 0.3-0.6 |
| Castor Oil | | | |
| Misc | add 100 | add 100 | add 100 |

“Misc” is understood to mean a cosmetic carrier, for example water and optionally additional conventional components of styling products.

### EXAMPLES

1. The following styling gels were produced:

| | Composition | | |
|---|---|---|---|
| | E1 | V1 | |
| Component/raw material | Wt. % | Wt. % | INCI name |
| Carbomer | 0.5 | 0.5 | Carbomer |
| 20000-30000 mPas (0.2%) | | | |
| XIAMETER ® OFX-0193 | 0.2 | 0.2 | PEG-12 |
| FLUID | | | Dimethicone |
| Aminomethyl ethanol | 0.34 | 0.34 | |
| D-panthenol | 0.1 | 0.1 | Panthenol |
| Luviskol ® K90 - 20% | 3 | 3 | |
| Advantage PG 30 | 10 | — | Methacryloyl ethyl |
| | | | betaine/acrylate |
| | | | copolymer |
| PVP/VA copolymer 60/40 | — | 6.48 | VP/VA copolymer |
| W NP | | | |
| Castor oil hydrog., 40 EO | 0.5 | 0.5 | PEG-40 |
| | | | Hydrogenated |
| | | | Castor Oil |
| L-arginine | 0.001 | 0.001 | |
| Nicotinic acid amide | 0.05 | 0.05 | |
| Uvinul MS 40 | 0.05 | 0.05 | Benzophenone-4 |
| Perfume | 0.3 | 0.3 | |
| Phenoxyethanol | 0.45 | 0.45 | |
| Ethanol | 10 | 10 | |
| Water | 100 | 100 | Water (aqua) |

The quantity specifications in the table specified as wt. % of the respective raw material, relative to the total agent. Styling gel E1 is an inventive agent for temporary deformation of keratinous fibers, while styling gel V1 is non-inventive.

Both hair gels formed a transparent film with very low stickiness after application to human hair.

Stiffness:

850 mg of a gel-like test composition was massaged into a dry hair strand (European natural hair from the company Kerling, 826500 thick glued extension, glued at one end, total length 150 mm, free length 130 mm, width 20 mm, weight 1.8±0.2 g) with the fingers. The hair strands treated with the test composition under examination were straightened in a Teflon rail with a diameter of 20 mm. The prepared strands were then dried and conditioned overnight at 21° C. and a relative humidity of 50% in an air-conditioned room.

The conditioned strand was carefully removed from the Teflon rail. The resulting flat strand was laid on the measuring blocks, which were spaced 40 mm apart. The 3PB adapter of an AMETEK LF Plus universal test instrument from the company AMETEK Precision Instruments Europe GmbH, product group Lloyd, was assembly centrally above the measuring blocks. The entire measurement process took place in the air-conditioned room under constant climatic conditions at 21° C. and 50% relative humidity.

To ensure standardized initial conditions, the measurement started with the application of a preload of 0.05 N. The strand was then compressed by 15 mm at a speed of 500 mm min⁻¹, wherein the required force was measured. The characteristic force K was then recorded when the maximum deformation of 15 mm was reached.

This method enables measurements on the basis of force Fₘₐₓ as a parameter of the stiffness of the polymer film and the degree of hold of the styling.

10 strands were prepared and measured for each test composition. Stiffness values ranging from about 3.2 to about 4.1 N were obtained. The average stiffness value of styling gel E1 was 3.67 N±0.6, and the average stiffness value of styling gel V1 was 2.85 N±0.4.

Long-Term Hold:

The shaping properties of the agent were examined by employing a long lasting hold measurement. To do so, standardized hair strands from the company Kerling (art. no. 826500) of hair type “European natural” color 6/0) with a length (Lₘₐₓ) of 220 mm and a weight of 3.0 g were used. In preparation, the strands were washed with a 12.5 wt. % sodium laureth sulphate solution. The hair strands were dried overnight at 45° C. in a drying oven.

The hair was soaked in lukewarm water for 20 minutes and then dabbed dry to a residual moisture content of approx. 50%.

750 mg of the agent was applied to each of the hair strands and massaged in. The hair strands were placed in a Teflon rail, smoothed using a steel roller and dried overnight at 21° C. and 50% relative humidity.

The hair strands were then clamped at one end in a holding device and stored at 21° C. and 85% relative humidity for a period of six hours. To calculate the long lasting hold (LLH), the lengths of the strands protruding from the holding device were measured before (L₀) and after (Lₜ) storage.

Long lasting hold is a measure of the temporal change in length of a stand of hair fixed using a hair shaping agent. The higher the LLH value, the smaller the change in length of hair strands under the influence of air humidity over a particular period of time and the better the degree of hold of the hair shaping agent.

The long lasting hold was calculated according to the following formula.

LLH=1−(Lₜ−L₀/Lₘₐₓ)

LLH values of from about 59-about 73% were determined for styling gel E1. LLH values of from about 8-about 10% were determined for styling gel V1.

Styling gel E1, therefore, demonstrated an exceptionally good combination of long-term hold and stiffness.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic agent for temporary deformation of keratinous fibers, which comprises:
(a) at least one copolymer A, comprising:
at least one monomer A1 selected from acrylic acid, methacrylic acid, acrylic acid alkyl esters and methacrylic acid alkyl esters, and
at least one amphoteric monomer A2 of the Formula (I) 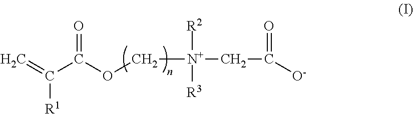
wherein R¹ for H or CH₃, R² and R³, each independently of one another, denote branched or non-branched C₁₋₁₀ alkyl, and n denotes an integer from about 1 to about 20, and
(b) at least one vinylpyrrolidone homopolymer.

2. Cosmetic agent according to claim 1, wherein the copolymer A comprises at least two different monomers A1.

3. Cosmetic agent according to claim 1, wherein the monomer A2 is selected from (meth)acryloyl alkyl betaines of the Formula (I), wherein R¹, R² and R³ denote CH₃, and n denotes 2.

4. Cosmetic agent according to claim 1, wherein the agent additionally comprises at least one thickening agent.

5. Cosmetic agent according to claim 1, wherein copolymer A (a) is a copolymer with INCI name methacryloyl ethyl betaine/acrylate copolymer, and the vinylpyrrolidone homopolymer (b) has a K value of from about 80 to about 100.

6. Cosmetic agent according to claim 1, which, relative to the total weight of the cosmetic agent, comprises:
from about 8 to about 12 wt. % of the copolymer A (a), and
from about 1 to about 5 wt. % of the vinylpyrrolidone homopolymer (b).

7. Cosmetic agent according to claim 1, wherein it additionally comprises from about 0.1 to about 0.5 wt. %—relative to the total agent—of aminomethyl propanol.

8. Cosmetic agent according to claim 1, wherein the cosmetic agent comprises less than 1 wt. %, relative to the total weight of the agent, of a vinylpyrrolidone comprising copolymer.

9. Use of a cosmetic agent according to claim 1 for temporary deformation of keratinous fibers.

10. Method for temporary deformation of keratinous fibers wherein the cosmetic agent according to claim 1 is applied on keratinous fibers.

11. Cosmetic agent according to claim 1, wherein the agent additionally comprises polyacrylic acid as a thickening agent.

12. Cosmetic agent according to claim 1, wherein the vinylpyrrolidone homopolymer (b) has a K value of about 90.
